(19) United States
(12) Patent Application Publication
(10) Pub. No.: US 2009/0061495 A1
(43) Pub. Date: Mar. 5, 2009

(54) Treatment Systems and Processes for Lignocellulosic Substrates that Contain Soluble Carbohydrates

(76) Inventors: Chris Beatty, Albany, OR (US); Steve Potochnik, Corvallis, OR (US); Grant Pease, Corvallis, OR (US)

Correspondence Address: WHITE-WELKER & WELKER, LLC, P.O. BOX 199, CLEAR SPRING, MD, 21722-0199, US

(21) Appl. No.: 12/199,794

(22) Filed: Aug. 27, 2008

Related U.S. Application Data
(60) Provisional application No. 60/969,374, filed on Aug. 31, 2007.

(51) Int. Cl. C12P 7/10

(52) U.S. Cl. 435/165

(57) ABSTRACT

A biorefining process used to convert lignocellulosic biomass into ethanol via a fermentation pathway. In a first pretreatment process step, the biomass is mixed with an aqueous wash solution to remove soluble carbohydrates from the biomass structure. Next, the solid fraction is separated from a liquid fraction. In a second pretreatment process, the solid fraction is pre-treated to make the fiber bundles and complex polysaccharides more amenable to enzymatic hydrolysis. Following the second pretreatment process, the pre-treated biomass is subjected to one or more enzymes in a hydrolysis process. The liquid fraction isolated from the first pretreatment process is diverted past the second pretreatment process and is recombined with the solid fraction in the hydrolysis process. The enzyme cocktail in the hydrolysis process breaks down the alpha- and hemicellulose polymers into fermentable sugars. Finally, a fermentation process produces a “beer” that is further processed in a distillation and dehydration process.

### CROSS REFERENCE TO RELATED APPLICATIONS

This application claims priority from U.S. Provisional Patent Application Ser. No. 60/969,374, entitled “Treatment Systems and Processes for Lignocellulosic Substrates that Contain Soluble Carbohydrates”, filed on 31 Aug. 2007. The benefit under 35 USC § 119(e) of the United States provisional application is hereby claimed, and the aforementioned application is hereby incorporated herein by reference.

### FEDERALLY SPONSORED RESEARCH

Not Applicable

### SEQUENCE LISTING OR PROGRAM

Not Applicable

### TECHNICAL FIELD

The present disclosure is directed to treatment systems and processes for lignocellulosic substrates, including pretreatment processes for removing soluble carbohydrates from a lignocellulosic biomass.

### BACKGROUND

Ethanol has become an increasingly important source for motor fuel and fuel additive. Biorefining processes which convert sugars and starches to ethanol via a fermentation pathway have long been used to produce ethanol for these fuels. Commonly used feedstocks for ethanol production include corn and sugarcane because they have accessible sugars and starches that are easily fermented into ethanol. Certain other biomass sources such as straw from grasses, fruit pomace (grapes, apples, citrus fruits, etc), artichokes, a variety of beans, sugar beet pulp and the like, which have both soluble carbohydrates and lignocellulosic fractions, are not typically utilized with the conventional sugar/starch enzymatic conversion processes because the soluble carbohydrate concentration on its own is too low to be economical. Typically, more complex processes are required to release usable sugars from these combination feedstocks. These complex processes are fairly expensive and do not provide as great of a yield as is desired.

### SUMMARY OF THE INVENTION

The present invention is a biorefining process used to convert lignocellulosic biomass into ethanol via a fermentation pathway in accordance with an embodiment of the present disclosure. In an optional mechanical reduction process step, the lignocellulosic feedstock is brought to a facility where it is mechanically reduced by chopping, milling, grinding, cutting, etc. In a first pretreatment process step, the biomass is mixed with an aqueous wash solution to remove soluble carbohydrates from the biomass structure. Following that process, the solid fraction is separated from a liquid fraction. In a second pretreatment process, the solid fraction is chemically and/or physically pre-treated to make the fiber bundles and complex polysaccharides more amenable to enzymatic hydrolysis. These secondary pretreatments are often quite harsh and can include one or more treatments with acids (sulfuric, nitric, hydrochloric), bases (NaOH, Na₂CO₃, NH₃), steam explosion or pressurized water, and/or strong solvents (acetone), etc. to form a biomass “slurry”.

Following the chemical, or otherwise harsh, second pretreatment process, the pre-treated biomass slurry is generally subjected to one or more enzymes (e.g., hydrolases) in a hydrolysis process. The liquid fraction isolated from the first pretreatment process is diverted past the second pretreatment process and is recombined with the solid fraction in the hydrolysis process. The enzyme cocktail used in the hydrolysis process, breaks down the alpha- and hemicellulose polymers into fermentable sugars.

Often the hydrolysis process is combined with a fermentation process. The fermentation process produces a “beer” that is further processed in a distillation and dehydration process. The “beer” created by the microorganisms is distilled and dehydrated in much the same way as is done in the grain/corn based ethanol processes in widespread use today.

A high percentage of the overall energy usage and capital cost in an ethanol plant occurs at the distillation and dehydration process. Energy usage and the associated costs are reduced with higher beer ethanol concentration. Additionally, higher beer concentration increases the throughput of ethanol for a given size of fermentation system and stipping column thus lowering capital costs per unit of ethanol produced. By selectively converting the soluble carbohydrates to ethanol, the process can increase fermentation and overall ethanol yield per unit feedstock. Additionally, because of the preliminary removal of the soluble carbohydrates prior to the second pretreatment process the fermenting microorganisms will not be subjected to the inhibitory byproducts from the chemical degradation of these sugars.

The biorefining process described herein is that soluble carbohydrates present in the biomass feedstocks are not subjected to the harsh and/or chemical treatments that result in toxic degradation products that inhibit fermenting microorganisms. Accordingly, by removing at least a portion of the soluble carbohydrates from the alpha- and hemicellulose and lignin-containing bundles prior to the harsh second pretreatment process, the fermentation process is more efficient and the process exacts a higher ethanol yield.

### BRIEF DESCRIPTION OF THE DRAWINGS

FIG. 1 is a schematic, cross-sectional view of cellulose fiber bundles found in lignocellulosic substrates.

FIG. 2 is a block diagram illustrating a biorefining process used to convert lignocellulosic biomass into ethanol via a fermentation pathway in accordance with an embodiment of the present disclosure.

FIG. 3 is a schematic diagram illustrating additional features of the biorefining process of FIG. 2 in accordance with an embodiment of the present disclosure.

FIG. 4 is a graph representing sugar accumulation as measured during consecutive batch recycling of the liquid fraction, and in accordance with an embodiment of the present disclosure.

FIG. 5 is a graph comparing soluble carbohydrate concentration in the liquid fraction to soluble carbohydrate retention in the solid fraction as measured for consecutive batches of recycled aqueous wash solution, and in accordance with an embodiment of the present disclosure.

### DETAILED DESCRIPTION

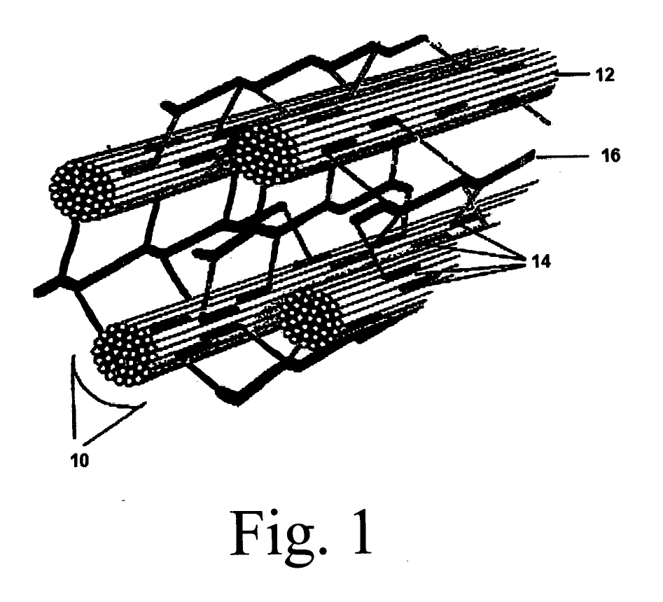

The present disclosure describes biorefining processes and systems, including processes and systems for converting lignocellulosic biomass into ethanol via a fermentation pathway. Several specific details of the disclosure are set forth in the following description and in FIGS. 1-5 to provide a thorough understanding of certain embodiments of the disclosure. One skilled in the art, however, will understand that the present disclosure may have additional embodiments, and that other embodiments of the disclosure may be practiced without several of the specific features described below.

FIG. 1 is a schematic, cross-sectional view of cellulose fiber bundles 10 found in lignocellulosic substrates, e.g., plants. Alpha-cellulose 12 is a linear polymer of glucose that is not soluble in water. Hemicellulose 14, which is also insoluble in water, is a branched polymer consisting of multiple pentose (C5) and hexose (C6) sugars depending on the starting feedstock material. Xylose (not shown), a C5 sugar, is commonly present in hemicellulose, while arabinose, mannose, glucose, and other sugars may also be present. Lignins 16 are a cross-linking polymer that acts like a glue to hold the fiber bundles 10 together.

Due to the increased cross-linking of the complex polysaccharides (e.g., alpha-cellulose 12 and hemicellulose 14 polymers), a lignocellulosic biorefining process requires the use of harsher treatments than biochemical processes used to convert only directly fermentable monomeric sugars and easily hydrolysable sugars, as well as a combination of selected hydrolases to break down the complex polysaccharides into fermentable sugars. These sugars may be either C6 or C5. Most C6 sugars are readily fermentable by conventionally used yeast populations, whereas most C5 sugars are not. Some wild yeast strains, as well as some genetically-modified yeast and bacteria strains, are able to metabolize C5 sugars, and use of these strains in fermentation processes is an evolving aspect of this biorefinement technology.

In addition to the complex polysaccharides (e.g., alpha-cellulose 12, hemicellulose 14), and lignin 16 illustrated in FIG. 1, several of the lignocellulosic feedstocks (e.g., fruit pomace, grasses, etc.) contain a significant fraction of their mass as soluble carbohydrates. These may be monomers such as glucose or fructose, or they may be oligomers or polymers of sugars such as fructo-oligosaccharides (FOS), inulin, starches, etc. For example, mature ryegrass (Lolium) has approximately 10 weight percent of its biomass stored as soluble carbohydrates, and greater than 10 weight percent if the plant is harvested at an earlier stage in the life cycle. The soluble carbohydrates in ryegrass are a combination of monomers (glucose, fructose), a dimer (sucrose), and oligomers in the inulin and levan series such as kestotriose, kestotetraose, etc.

Lignocellulsoic feedstocks, including those with soluble carbohydrates, are not heavily utilized for ethanol production due to the cost of enzymes and capital equipment. Specifically, the soluble carbohydrates in feedstocks can include sugar monomers or sugars that are easily hydrolyzed to monomers. Relatively harsh pretreatment processes (e.g., pH<2, pH>11, oxidizing agents, high temperatures, rapid phase change) used to disrupt the fiber bundles 10 and complex polysaccharides can further degrade the soluble carbohydrates to toxic compounds that are inhibitory to the microorganisms (e.g., yeast and bacteria) used to ferment the hydrolyzed sugars. A representative group of these inhibitory compounds are known as furans. Common examples of furans include furfural and hydroxymethylfurfural. These toxic compounds reduce both the productivity (in grams of ethanol produced per gram of sugar per hour) and final ethanol yield of the process (grams of ethanol produced per gram of sugar or raw biomass input).

Lignocellulosic feedstocks containing a significant fraction of their mass as soluble carbohydrates (e.g., grasses, fruit pomace, artichokes, a variety of beans, sugar beet pulp, etc.), provide a resource that can be used for the production of ethanol. For example, in addition to the alpha- and hemicellulose 12 and 14, and the lignin 16 molecules being used to produce ethanol, many soluble carbohydrates (glucose, sucrose, fructose, etc.) are directly usable by the microorganisms and/or are readily hydrolyzed by milder enzymatic or inorganic processes to fermentable sugars.

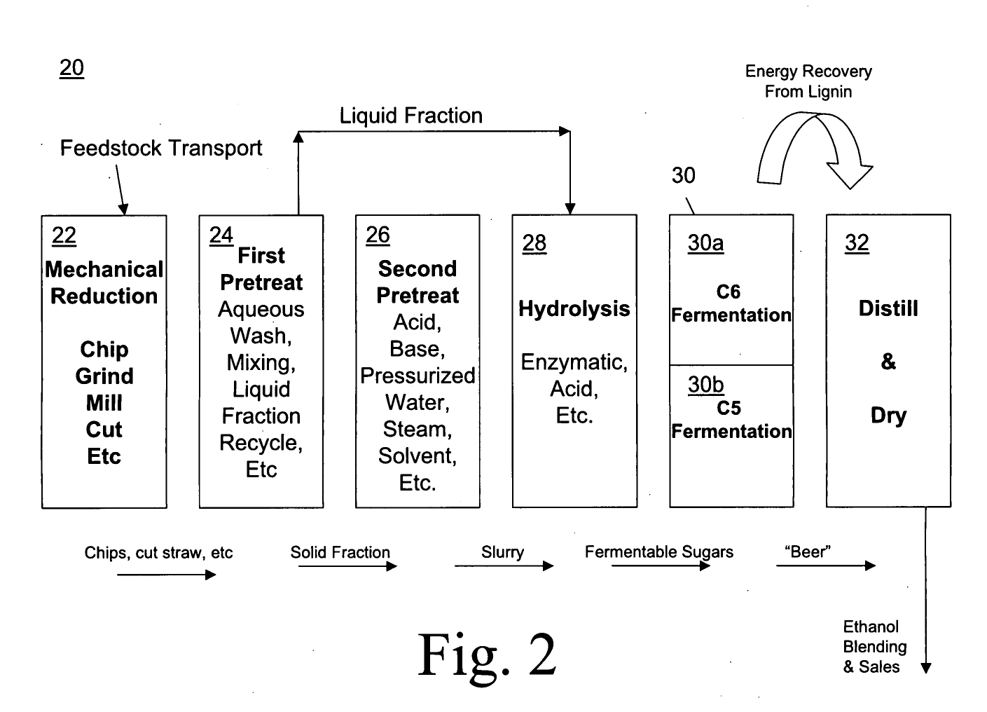

FIG. 2 is a block diagram generally illustrating a biorefining process 20 used to convert lignocellulosic biomass into ethanol via a fermentation pathway in accordance with an embodiment of the present disclosure. In an optional mechanical reduction process step 22, the lignocellulosic feedstock is brought to a facility where it is mechanically reduced by chopping, milling, grinding, cutting, etc. Certain feedstocks, such as reject material from seed cleaning operations or some fruit pomace may not require mechanical reduction. In a first pretreatment process step 24, the biomass from step 22 (often mechanically reduced biomass) is mixed with an aqueous wash solution to remove soluble carbohydrates from the biomass structure. Following process step 24, the solid fraction is separated from a liquid fraction. In a second pretreatment process step 26, the solid fraction is chemically and/or physically pre-treated to make the fiber bundles 10 and complex polysaccharides more amenable to enzymatic hydrolysis. These secondary pretreatments are often quite harsh and can include one or more treatments with acids (sulfuric, nitric, hydrochloric), bases (NaOH, Na₂CO₃, NH₃), steam explosion pressurized hot water, and/or strong solvents (acetone), etc. to form a biomass “slurry”.

Following the chemical, or otherwise harsh, second pretreatment process step 26, the pre-treated biomass slurry is generally subjected to one or more enzymes (e.g., hydrolases) in a hydrolysis process step 28. The liquid fraction isolated from the first pretreatment process step 24 is diverted past the second pretreatment process step 26 and is recombined with the solid fraction in the hydrolysis process step 28. The enzyme cocktail used in the hydrolysis process step 28, breaks down the alpha- and hemicellulose polymers 12 and 14 into fermentable sugars. Suitable enzymes can include cellulase, cellobiose dehydrogenase, xylosidase, etc. Cocktails of suitable enzymes can be purchased from Novozymes of Bagsvaerd, Denmark. In another embodiment, however, other techniques, such as acid hydrolysis, can be used to break down alpha- and hemicellulose 12 and 14 into fermentable sugars.

Often the hydrolysis process step 28 is combined with a fermentation process step 30 that includes either a C6 fermentation step 30a or both the C6 and a C5 fermentation steps 30a and 30b into a single fermentation process step. A variety of microorganisms can be utilized for fermentation such as wild and genetically-modified yeast and bacterial strains. The fermentation process step 30 produces a “beer” that is further processed in a distillation and dehydration process step 32. “Beer” can be simply defined as a mix of ethanol, water, and other organics produced by the fermenting of carbohydrates. The “beer” created by the microorganisms is distilled and dehydrated in much the same way as is done in the grain/corn based ethanol processes in widespread use today.

A high percentage of the overall energy usage and capital cost in an ethanol plant occurs at the distillation and dehydration process step 32. Energy usage and the associated costs are reduced proportionately with higher beer ethanol concentration. Additionally, higher beer concentration increases the throughput of ethanol for a given size of fermentation system and stipping column thus lowering capital costs per unit of ethanol produced. By selectively converting the soluble carbohydrates to ethanol, the process can increase fermentation and overall ethanol yield per unit feedstock. Additionally, because of the preliminary removal of the soluble carbohydrates prior to the second pretreatment process step 26, the fermenting microorganisms will not be subjected to the inhibitory byproducts from the chemical degradation of these sugars.

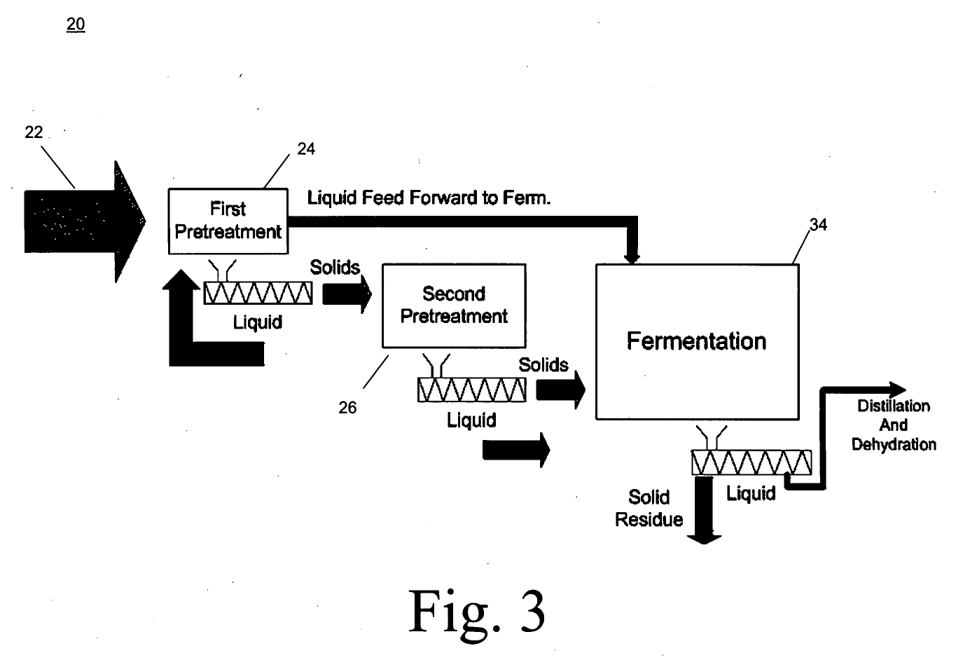

FIG. 3 is a schematic diagram illustrating additional features of the biorefining process 20 of FIG. 2 in accordance with an embodiment of the present disclosure. The biorefining process 20 often begins with the mechanical reduction process step 22 as described above. The biorefining process 20 further includes the first pretreatment process step 24 that removes at least a portion of soluble carbohydrates from the starting biomass.

In one embodiment, the first pretreatment process step 24 can be performed in a batch mode, wherein single batches of feedstock are processed sequentially. For example, a portion of incoming biomass may go through a mechanical reduction process step 22 and then is combined with an aqueous wash solution for a mixing period. The aqueous wash solution can be warm or hot. For example, the aqueous wash solution can have a temperature greater than an ambient temperature (e.g., approximately 25° C. to approximately 100° C.). An elevated temperature (e.g., a temperature greater than ambient temperature) can be beneficial for bringing and retaining soluble carbohydrates in solution. Temperatures near 100° C. may also have the beneficial effect of killing many native microorganisms that may convert sugars to undesirable products other than ethanol. In other arrangements, the temperature of the aqueous wash solution may not be elevated or otherwise heated. Additionally, the aqueous wash solution can have a neutral pH (e.g., approximately pH 5-pH 9). However, in another embodiment, the pH can be slightly acidic (e.g., approximately pH 2-pH 5). The lower pH may have the beneficial effect of hydrolyzing oligo- or polysaccharides such as inulin or levan to fermentable monomeric sugars. In some embodiments, the aqueous wash solution, once combined, can include about 5% to 20% mechanically reduced biomass by weight. In other embodiments, the biomass can be less than 5% or greater than 20% of the combined solution by weight.

The portion of biomass can be mixed in the aqueous wash solution using a mixing apparatus, such as a screw wash reactor, to facilitate the removal of soluble carbohydrates from the biomass, and thereby, release them into the aqueous wash solution. In other embodiments, the mixing apparatus can be another motor-driven paddle mixer or agitator. The batch can be mixed for a short period of time (e.g., approximately 10 minutes to approximately 60 minutes). In other arrangements, the batch can be mixed for a time shorter than 10 minutes or longer than 60 minutes.

Referring to FIG. 3 and following the mixing step of the first pretreatment process step 24, a solid biomass fraction is separated from at least a portion of a liquid fraction having at least a portion of the soluble carbohydrates using a press (e.g., a screw press, a filter press, etc.). In other embodiments, the solid fraction can be separated from the liquid fraction using a filter or a centrifuge. The solid fraction is fed forward to the second pretreatment process step 26 that can include one or more chemical/harsh treatments for disrupting the fiber bundles 10 and complex polysaccharides to make them more amenable to enzymatic hydrolysis and fermenting microorganisms in later process steps. As described above, hydrolysis and fermentation processes can occur simultaneously in a single hydrolysis/fermentation process step 34. In other arrangements, however, hydrolysis and fermentation of the sugars may occur separately.

In the batch mode, the liquid fraction can be recycled and used in subsequent rounds of the first pretreatment process step 24 prior to diverting the liquid fraction to the hydrolysis/fermentation process step 34. In this specific embodiment, the liquid fraction, having a first concentration of soluble carbohydrates, is mixed with additional batches or portions of biomass to increase the concentration of soluble carbohydrates present in the liquid fraction from the first concentration to a second concentration greater than the first concentration. The liquid fraction can be re-used to facilitate the removal of soluble carbohydrates from a plurality of biomass batches, e.g., approximately 1-10 batches. In other embodiments, the liquid fraction can be used in more than 10 batches. In some embodiments, additional aqueous wash solution can be added to the liquid fraction to increase the volume and/or replace lost volume from the liquid fraction due to partial retention (“drag-out”) by the solid fraction.

In one embodiment, the liquid fraction, having a sufficient concentration of soluble carbohydrates, is routed to the hydrolysis/fermentation process step 34. In some embodiments, the liquid fraction can be routed following completion of a predetermined number of batches. This number of batches can be determined by a number of process variables, such as the starting biomass, the temperature of the aqueous wash solution, the percent of the liquid fraction/soluble carbohydrates being lost to drag-out in the system, etc. In other arrangements, the percent weight of soluble carbohydrates in the liquid fraction can be tested periodically (e.g., by liquid chromatography, etc.) until an acceptable concentration is achieved. In the specific embodiment illustrated in FIG. 3, the liquid fraction is recombined with the chemically treated molecules of the solid fraction. Following hydrolysis and fermentation, the fermented solution is distilled and dehydrated in further process steps (e.g., process step 32 illustrated in FIG. 2). In some arrangements, remaining solid residue can be removed from the fermented solution prior to distillation and dehydration.

The liquid fraction can be recycled and mixed with additional batches of biomass while maintaining an ambient to hot temperature (e.g., approximately 25° C. to approximately 100° C.). In these arrangements, the liquid fraction can be routed to the hydrolysis/fermentation process step 34 prior to cooling below a suitable temperature for hydrolysis/fermentation. In some arrangements, the mixing apparatus and/or the separation apparatus can be heated to at least partially prevent cooling of the liquid fraction. In other embodiments, however, the liquid fraction can be heated and/or reheated as necessary to maintain a suitable temperature for carbohydrate solubility. For example, additional hot aqueous wash solution can be added to a cooled liquid fraction. In another example, the liquid fraction can be passed through hot steam. One of ordinary skill in the art will recognize a variety of techniques that can be used to maintain the temperature of and/or reheat the liquid fraction to promote carbohydrate solubility.

In another embodiment, the first pretreatment process step 24 can be performed in a continuous mode. In continuous mode operation, the biomass (often mechanically reduced) is mixed in the aqueous wash solution, as previously described, to facilitate the removal of at least a portion of the soluble carbohydrates from the biomass, releasing them into the aqueous wash solution. The solid fraction is separated from at least some of the liquid fraction, for example with a press, centrifuge, or filter. A substantial portion of the liquid fraction is recycled back into the mixing apparatus to concentrate the solution with additional soluble carbohydrates, as more biomass is deposited. In contrast to batch mode operation, a sub-portion of the liquid fraction is routed directly to the hydrolysis/fermentation process step 34. In other arrangements, the sub-portion of the liquid fraction can be routed to a storage area (not shown). Moreover, additional aqueous wash solution can be fed into the process (generally into the mix apparatus) to replace a volume substantially equal to the sub-portion volume diverted to the hydrolysis/fermentation process step 34. Additional volume of aqueous wash solution can be added during the biorefining process 20 to replace liquid volumes lost due to drag-out (e.g., unseparated liquid remaining with the solid fraction), or lost for other reasons.

In a further embodiment, the first pretreatment process step 24 can be performed in a semi-continuous mode. In semi-continuous mode operation, the mixture of the often mechanically reduced biomass and aqueous wash solution is mixed for a pre-set time period and separated as described above with respect to batch mode operation. After each separation interval, a percentage of the liquid fraction is diverted to the hydrolysis/fermentation process step 34, while the remainder is recycled and mixed with additional incoming biomass. In some embodiments, additional aqueous wash solution can be added to the mixing apparatus.

Operation parameters of all modes (e.g., batch, continuous, semi-continuous) are configured to divert the liquid fraction to the hydrolysis/fermentation process step 34 when the concentration of desirable soluble carbohydrates is sufficiently high enough to be useful to increase the final beer concentration. In some arrangements the biorefining process 20 can include an evaporation step (not shown). In these embodiments, a portion of the liquid fraction can be evaporated, further increasing the percent by weight concentration of soluble carbohydrates, prior to routing the liquid fraction to the hydrolysis/fermentation process step 34. In other arrangements, a sugar-selective membrane configured to either a) retain, or b) transport sugars can be employed to increase the percent by weight concentration of the sugars.

As mentioned above, a small portion of the liquid fraction is retained with the solid fraction following separation (i.e., drag-out). As the concentration of soluble carbohydrates in the liquid fraction increases with re-use in the first pretreatment process step 24, the percentage of soluble carbohydrates in the drag-out volume also increases. Inhibitory compounds can be generated in the second pretreatment process step 26 from these “lost” soluble carbohydrates and have detrimental effects during fermentation. Additionally, the biorefining process 20 loses the benefit of converting these lost sugars to ethanol. Therefore, the operation parameters (e.g., number of recycle occurrences for the liquid fraction, aqueous wash solution temperature and pH, first pretreatment mixing time, etc.) must also be set to limit major losses of soluble carbohydrates due to drag-out of the liquid fraction with the separated solid fraction.

In some arrangements, the solid fraction can be at least partially rinsed (e.g., counter-current rinsed) with additional aqueous wash solution or fresh “make-up” water (i.e. water used to compensate for drag-out after pressing/filtering) prior to the second pretreatment process step 26 to recover “lost” soluble carbohydrates. In one embodiment, the counter-current rinse solution can be combined with the liquid fraction following the rinsing process. In another embodiment, the counter current rinse solution can be diverted to the hydrolysis/fermentation process step 34.

In one embodiment, the harsh pretreatment liquids (e.g., acids, bases, water, solvents, ammonium hydroxide, etc.) from the second pretreatment process step 26 can be removed from the pretreated solid fraction and disposed of prior to transferring the solid fraction to the hydrolysis/fermentation process step 34. In this embodiment, inhibitory compounds generated from the degradation of soluble carbohydrates remaining with the solid fraction or in drag-out liquid, can be eliminated prior to fermentation.

In another embodiment, the harsh pretreatment liquids can be added to the fermentation broth if the second pretreatment process step 26 did not create substantial inhibitory compounds or if the liquid can be de-toxified in an economical manner. For example, the harsh pretreatment liquids can be neutralized; treated with lime to precipitate CaSO₄ and inhibitory compounds; further oxidized with addition O₂, O₃, H₂O₂, etc.; and the like. Accumulation of inhibitory compounds can be monitored using UV spectrometry. In a specific example, the inhibitory compound, furfural absorbs light and can be quantified in the 280 nm absorbance range. Accordingly, as inhibitory compounds rise to detrimental levels, as detected by UV spectrometry, operation parameters (e.g., number of recycle occurrences for the liquid fraction, harsh pretreatment liquid disposal, aqueous wash solution temperature and pH, first pretreatment mixing time, etc.) can be altered.

One feature of the biorefining process 20, operated in either continuous mode, semi-continuous mode, or batch mode as described above, is that the process concentrates levels of soluble carbohydrates to levels usable for fermentation. For example, by concentrating the levels of soluble carbohydrates, excess liquid is avoided in the fermentation system and stipping column resulting in higher beer concentration and more efficient ethanol production per biomass unit.

Another feature of the biorefining process described herein is that soluble carbohydrates present in the biomass feedstocks are not subjected to the harsh and/or chemical treatments that result in toxic degradation products that inhibit fermenting microorganisms. Accordingly, by removing at least a portion of the soluble carbohydrates from the alpha- and hemicellulose 12 and 14, and lignin-containing bundles 10 prior to the harsh second pretreatment process step 26, the fermentation process is more efficient and the process 20 exacts a higher ethanol yield.

The following examples are provided by way of illustration, and are not intended to be limiting of the present disclosure.

### EXAMPLE I

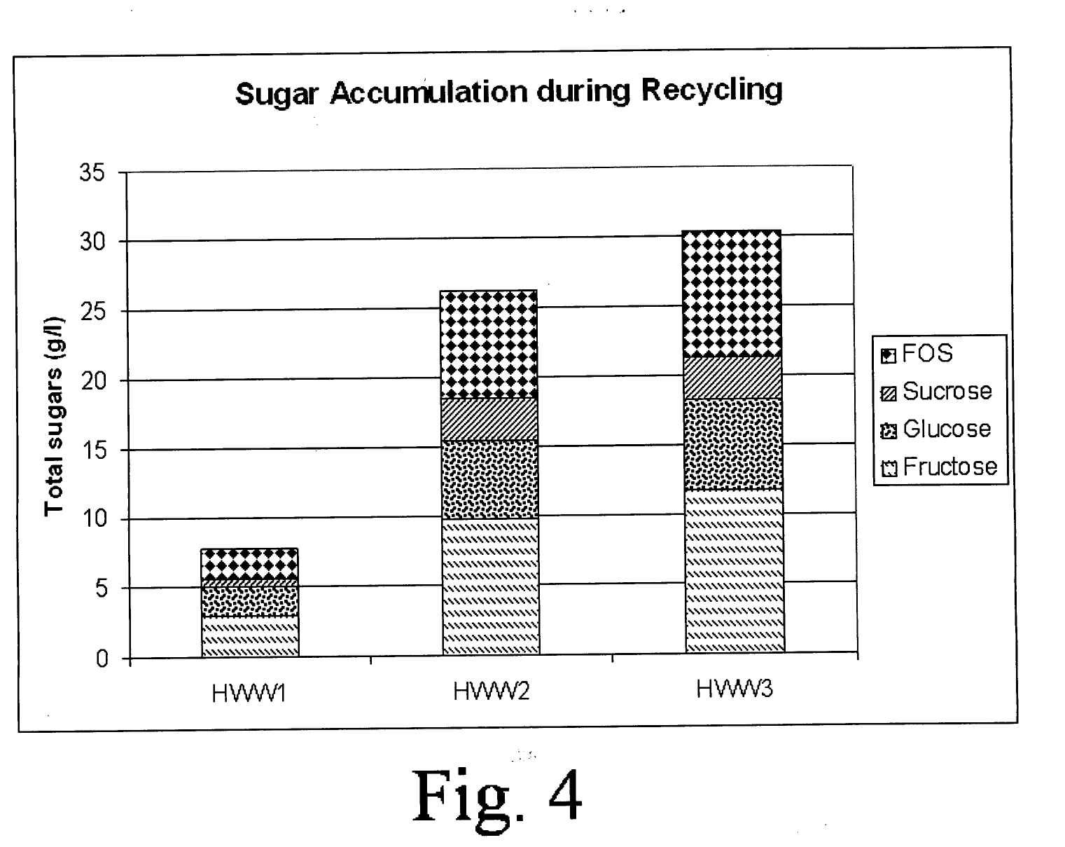

FIG. 4 is a graph representing sugar accumulation as measured during consecutive batch recycling of the liquid fraction, and in accordance with an embodiment of the present disclosure. The biorefining process 20 (illustrated in FIGS. 2 and 3) was operated in batch mode on ryegrass straw. The liquid fraction was collected from a first batch and a first batch sample was taken. The liquid fraction was recycled and used to collect soluble carbohydrates from second and third batches. Second and third batch samples were taken following the second and third batches, respectively. The first, second and third batch samples were analyzed by liquid chromatography to determine the concentrations of soluble carbohydrates (e.g., fructose, glucose, sucrose, fructo-oligosaccharides [FOS]) in the respective batch samples. Referring to FIG. 4, the liquid chromatography data show an increase in concentration for all soluble sugars during successive batches.

### EXAMPLE II

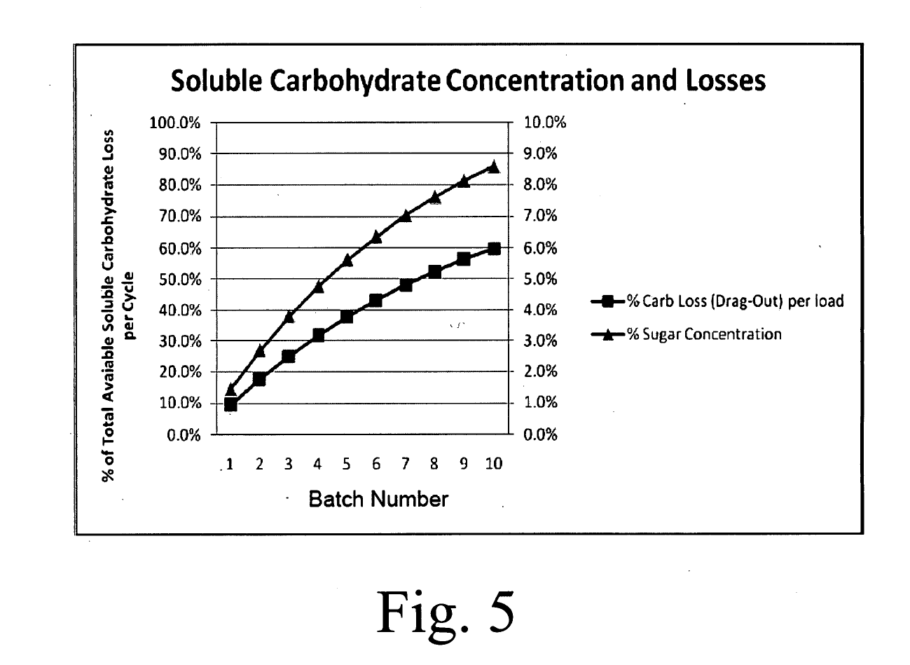

FIG. 5 is a graph comparing soluble carbohydrate concentration in the liquid fraction to soluble carbohydrate retention in the solid fraction as calculated for consecutive batches of recycled aqueous wash solution, and in accordance with an embodiment of the present disclosure. As the model shows in FIG. 5, the soluble carbohydrate concentration substantially increases as the liquid fraction is recycled during batch mode operation. The model also shows that while the carbohydrate concentration increases the soluble carbohydrate loss due to “drag-out” (e.g., liquid fraction retention, unreleased soluble carbohydrates, etc.) increases as well.

For a given biomass conversion facility, optimum operating conditions (e.g., the amount of recycled liquid, the rinse temperature, the rinse chemistry, etc.) for the first pretreatment process step 24 can be determined by considering some or all of the following: 1) minimizing losses of usable soluble carbohydrates through “drag-out” in the subsequent solid fraction separation step, 2) minimizing detrimental effects to ethanol yield due to soluble carbohydrate degradation into microorganism inhibitors, and 3) maximizing the final beer concentration. These operating conditions may be determined prior to running the biorefining process 20, or a control loop may be developed by one of ordinary skill in the art to adjust the operating parameters in real time based on certain past, current, and/or predicted future conditions of the feedstock, the first pretreatment, or subsequent, processing steps. For example, the soluble carbohydrate level in the first pretreatment process step 24 may be monitored and used as a control signal in a feedback loop to optimally adjust the amount of separated liquid fraction that is returned to the mixing apparatus. Additionally, differences in feedstock will have varying concentrations and types of soluble carbohydrate, and the biorefining process 20 would optimally change the relative amounts of separated liquid fraction (or number of “batch” mode rinses) recycled back to the first pretreatment process step 24, as well as other mixing process parameters such as agitation speed, pressure, temperature, and chemical composition of the mixture.

From the foregoing, it will be appreciated that specific embodiments of the disclosure have been described herein for purposes of illustration, but that various modifications may be made without deviating from the spirit and scope of the invention. Accordingly, the disclosure is not limited except as by the appended claims.

I/We claim:

1. A process for treating a biomass to maximize ethanol yield, comprising the steps of:
(a) in a first pretreatment, the biomass is mixed with an aqueous wash solution to remove soluble carbohydrates from the biomass;
(b) a solid fraction is separated from a liquid fraction;
(c) in a secondary pretreatment, the solid fraction is pre-treated to make the fiber bundles and complex polysaccharides more amenable to enzymatic hydrolysis;
(d) a liquid fraction isolated from the first pretreatment process step (a) is diverted past the second pretreatment process step (c) and is recombined with the solid fraction to create a slurry;
(e) the pre-treated biomass slurry is subjected to one or more enzymes breaking down the alpha- and hemicellulose polymers into fermentable sugars in a sugar solution; and
(f) fermenting the sugar solution to an ethanol solution.

2. The process according to claim 1, wherein the biomass is mechanically reduced before the first pre-treating process of step (a).

3. The process according to claim 1, wherein the biomass is washed in a batch fashion.

4. The process according to claim 3, wherein some of the wash liquor is recycled to concentrate the soluble carbohydrates.

5. The process according to claim 1, wherein the biomass is washed in a continuous fashion.

6. The process of claim 5, wherein fresh or recycled water flows counter current to the biomass.

7. The process according to claim 1, wherein the secondary pretreatments of step (c) includes one or more treatments with acids, bases, steam explosion and/or pressurized hot water, and/or strong solvents.

8. The method of claim 1, further comprising the steps of:
(g) removing any harsh pretreatment liquids from the second pretreatment process of step
(c) from a pretreated solid fraction produced by step (c); and
(h) disposing of the harsh pretreatment liquids prior to transferring the solid fraction produced by step (d) to the hydrolysis/fermentation process of step (e).

9. The method of claim 1, further comprising the step of:
(j) adding harsh pretreatment liquids to the hydrolysis/fermentation broth of step (e).

10. The process according to claim 1, wherein suitable enzymes for use in step (e) include cellulase, cellobiose dehydrogenase, and xylosidase/hemicellulase.

11. The process according to claim 1, further comprising the step of:
(k) measuring the amount of one or more monomeric sugars in the liquid fraction during or after process step (a) and before process step (d).

12. The process of claim 1, wherein the hydrolysis and fermentation of the biomass in steps (e) and (f) occur separately.

13. The method of claim 1, wherein the hydrolysis and fermentation of the biomass in steps (e) and (f) occur simultaneously.

14. The process according to claim 1, further comprising the steps of:
(l) wherein the ethanol solution of step (f) is further processed in a distillation and dehydration process.

15. The process according to claim 1, wherein the fermentation process of step (f) is further comprised of a plurality of fermentation steps.

16. A method for a biorefining process comprising the steps of:
(a) subjecting the biomass to a first pretreatment process step that removes at least a portion of soluble carbohydrates from the starting biomass;
(b) separating a solid biomass fraction from at least a portion of a liquid fraction having at least a portion of the soluble carbohydrates;
(c) feeding the solid fraction to a second pretreatment process step;
(d) subjecting the combined pretreated solid biomass and some or all of the liquid fraction from (a) to a hydrolysis and fermentation process; and
(e) distilling and dehydrating a fermented solution.

17. The method of claim 16, wherein the first pretreatment process is performed in a batch mode, further comprising the steps of:
(f) processing single batches of feedstock sequentially; and
(g) recycling a liquid fraction to be used in subsequent rounds of the first pretreatment process step (a) prior to diverting the liquid fraction to the hydrolysis/fermentation process step (d).

18. The method of claim 16, wherein
a portion of incoming biomass goes through a mechanical reduction process and then is combined with an aqueous wash solution for a mixing period;
the aqueous wash solution has a temperature between approximately 25° C. to approximately 100° C.; and
the aqueous wash solution has a neutral pH of about 5 to about 9.

19. The method of claim 18, wherein the aqueous wash solution has an acidic pH of about 2 to about 5.

20. The method of claim 18, wherein the aqueous wash solution includes about 5% to 20% mechanically reduced biomass by weight.

21. The method of claim 18, wherein the aqueous wash solution includes about 1% to 5% mechanically reduced biomass by weight.

22. The method of claim 18, wherein the aqueous wash solution includes about 20% to 50% mechanically reduced biomass by weight.

23. The method of claim 15, wherein a press, filter, or centrifuge is used to separate a solid biomass fraction from at least a portion of a liquid fraction having at least a portion of the soluble carbohydrates in step (b).

24. The method of claim 16, wherein step (c) is further comprised of one or more chemical/harsh treatments for disrupting fiber bundles and complex polysaccharides in the biomass.

25. The method of claim 16, wherein the hydrolysis and fermentation of the biomass in step (d) occur separately.

26. The method of claim 16, wherein the hydrolysis and fermentation of the biomass in step (d) occur simultaneously.

27. The method of claim 17, wherein the liquid fraction from step (b) is routed following completion of a predetermined number of batches.

28. The method of claim 17, wherein a fraction of liquid fraction which is recycled to step (b) and the fraction that is routed to process step (d) are determined in whole or in part by measuring one or more monomeric sugars in the liquid fraction during or after process step (a) or process step (b).

29. The method of claim 17, wherein the liquid fraction from step (b) is recycled and mixed with additional batches of biomass while maintaining a temperature of approximately 25° C. to approximately 100° C.

30. The method of claim 16, wherein the first pretreatment process of step (a) is performed in a continuous mode wherein the biomass is mixed in an aqueous wash solution to facilitate the removal of at least a portion of the soluble carbohydrates from the biomass, releasing them into the aqueous wash solution, and a sub-portion of the liquid fraction from step (b) is routed directly to the hydrolysis/fermentation process of step (d).

31. The method of claim 30, wherein the sub-portion of liquid fraction from step (b) which is routed to the process step (d) is determined in whole or in part by measuring the content of one or more monomeric sugars in the liquid during or after process step (a) or process step (b).

32. The method of claim 17, wherein the first pretreatment process of step (a) is performed in a semi-continuous mode wherein the biomass and aqueous wash solution is mixed for a pre-set time period and separated with respect to batch mode operation so that after each separation interval, a percentage of the liquid fraction from step (b) is diverted to the hydrolysis/fermentation process of step (d), while the remainder is recycled and mixed with additional incoming biomass.

33. The method of claim 32, further comprising the step of
(h) evaporating a portion of the liquid fraction from step (b), further increasing the percent by weight concentration of soluble carbohydrates, prior to routing the liquid fraction from step (b) to the hydrolysis/fermentation process of step (d).

34. The method of claim 16, further comprising the step of:
(i) partially rinsing the solid fraction from step (b) with an additional aqueous wash solution prior to the second pretreatment process of step (c).

35. The method of claim 34, wherein the solution is either combined with the liquid fraction of step (b) following the rinsing process or diverted to the hydrolysis/fermentation process of step (d).

36. The method of claim 16, further comprising the steps of:
(j) removing any harsh pretreatment liquids from the second pretreatment process of step (c) from a pretreated solid fraction produced by step (c); and
(k) disposing of the harsh pretreatment liquids prior to transferring the solid fraction produced by step (c) to the hydrolysis/fermentation process of step (d).

37. The method of claim 16, further comprising the step of:
(l) adding harsh pretreatment liquids to the fermentation broth of step (d).
